(19) United States
(12) Patent Application Publication
(10) Pub. No.: US 2004/0092592 A1
(43) Pub. Date: May 13, 2004

(54) Stabilized L-Arginine platelet aggregation inhibitory compositions and processes for making same

(76) Inventor: Leonard L. Kaplan, East Brunswick, NJ (US)

Correspondence Address: Leonard L. Kaplan, Ph.D, One Minute Man Court, East Brunswick, NJ, 08816, US

(21) Appl. No.: 10/291,283

(22) Filed: Nov. 8, 2002

(51) Int. Cl. A61K 31/60; A61K 31/50; A61K 31/4743; A61K 31/198

(52) U.S. Cl. 514/565; 514/165; 514/247; 514/301

(57) ABSTRACT

Linking Magnesium ions to Nitric Oxide precursor L-Arginine, chemically 2-amino-5-guanidino valeric acid, with a platelet aggregation inhibitor compound such as, but not limited to acetylsalicylic acid or clopidogrel bisulfate, unexpectedly results in a pharmaceutically stabilized compositions with extended shelf life to be taken orally to provide gradual release vasodilatory and anti-platelet aggregation pharmacological activity with reduced potential for producing gastrointestinal lesions. L-Arginine releases ADNO (Arginine derived Nitric Oxide) in the coronary artery epithelium as EDRF (endothelium dependent relaxing factor) to dilate the arteries to promote blood flow to the myocardium, and the platelet aggregation inhibitor such acetylsalicylic acid or clopidogrel and others of this class of drugs inhibits or antagonizes the aggregation adhesion of platelets in the blood stream. Aggregated or clumped blood platelets contribute to arterial stenosis due to formation of atherosclerotic plaques that occlude coronary and other circulatory arteries. In addition to coronary arteries, atherosclerotic plaques can occlude and stenose carotid arteries and femoral arteries due to aggregated or clumped blood platelets, and the subject of this patent discovery will also be of cardiovascular health benefit respectively in preventing carotid cerebrovascular accidents and femoral artery leg circulation disease.

### BACKGROUND OF THE INVENTION

There is a medical need to provide cardiovascular patients suffering from atherosclerotic disease with an oral medicine to dilate blocked arteries and improve blood flow through the coronary arteries, the carotid arteries and the femoral arteries to the heart, the brain, and the leg musculature respectively. Current state of the art consists of organic nitrate drugs to provide some vasodilation activity and other drugs none as “blood thinners” to inhibit blood platelet aggregation in circulatory atherosclerotic plaques. However, it has not been technically feasible to combine these two classes of pharmacological agents due to pharmaceutical acid-base instability problems. This invention relates to oral pharmaceutical compositions to increase arterial blood flow consisting of the basic amino acid L-Arginine that releases nitric oxide, the epithelial derived relaxation factor to dilate coronary and other body circulation arteries combined with acid pH platelet adhesion inhibitor drug(s) including acetylsalicylic acid, clopidogrel bilsufate and others in this pharmacological class of drugs, stabilized with a divalent magnesium ion salt such as, but not limited to magnesium carbonate to provide gradual release of the drugs; to lower potential for gastrointestinal lesions; and to extend the shelf life of the acid-base physicochemically unstable compositions wherein the said stabilized subject compositions provide a preferred method to prevent and treat cardiovascular, cerebrovascular, and leg arterial circulation occlusion diseases.

### DESCRIPTION OF RELATED ART

Cardiovascular, cerebrovascular, and leg circulation occlusion diseases occur when arteries become narrowed or occluded (known medically as stenosis) to the point where there is a reduced blood supply to the heart, brain and leg musculature respectively. Platelets are microscopic oval discs that circulate in the blood stream and normally serve an important function in blood clotting. However, individuals with high cholesterol tend to form arterial atherosclerotic plaques in which platelets also play a role to block normal blood flow through the arteries. Normally, platelets carry a negative charge to prevent clumping and adhering to arterial walls, but in cases of atherosclerosis, the platelets adhere to the cholesterol formed plaques that block the normal blood flow resulting in ischemia or blood deficiencies in the related organs. In the heart, blockage of the coronary arteries can result in infarcts or damaged heart muscle resulting in a heart attack; in the brain, blockage of the carotid arteries can result in strokes due to reduced oxygen supply to the brain; and in the legs, blockage of the femoral arteries can result in damage to the upper and lower leg muscles.

Several chemical substances have been medically used to provide an oral source of nitric oxide to the arterial circulation to relax the smooth musculature of arteries and maintain patency of the body's arteries. Among these are isosorbide mono and dinitrate, erythritol tetranitrtate, and L-Arginine. Chemically, L-Arginine is 2-amino-5-guanidino valeric acid, a highly basic amino acid, and is a preferred embodiment of this invention in that it serves as a dietary supplement source of nitric oxide in the body wherein L-Arginine is acted upon by the enzyme nitric oxide synthase to form nitric oxide. Nitric oxide has been shown clinically, as reported in the above cited referenced publications and patents, to effect arterial endothelium relaxation and dilation. This nitric oxide metabolite is known functionally as “EDRF”, the Endothelial Derived Relaxation Factor, and is well described in the cited literature and patent references. Medically, this amino acid L-Arginine plays an important beneficial cardiovasular health role to maintain the blood flow in the arterial circulation. Another separate group of drug substances known as “blood thinners” have been demonstrated clinically to prevent adhesion, clumping or aggregation of blood platelets, which in contact with elevated cholesterol levels in the blood, will form atherosclerotic plaques that block the normal flow of blood. The pathologic results of the resultant blockage or stenosis of the arteries are heart attacks in the case of coronary artery blockage; cerebrovascular strokes in the case of carotid artery blockage; and severe leg muscle damage in the case of femoral artery circulation blockage. Examples of these “blood thinner” drugs that inhibit the adhesion, clumping and aggregation of blood platelets are, but not limited to, acetylsalicylic acid, clopidogrel bisulfate, ticlopidine hydrochloride, anagrelide hydrochloride, pyridimole hydrochloride and the like. One type of “blood thinner” drugs typified by acetylsalicylic acid, serve as prostaglandin synthetase inhibitors, thereby preventing blood platelet aggregation and clotting by inhibiting the prostaglandin synthetase Thromboxane A2 (TxA2), the prostaglandin enzyme required for blood platelet aggregation and clotting. Another type of “blood thinner” drugs typified by clopidogrel bisulfate, ticlopidine hydrochloride and anagrelide hydrochloride serve as inhibitors of adenosine diphosphate (ADP) induced plataelet aggregation by binding to the ADP receptor sites thereby interfering with ADP-collagen mediated platelet aggregation. Both these pharmacological class of “blood thinner” drugs are FDA approved as effective medically, and are marketed individually as commerical pharmaceutical products for the indicated cardiovascular health ben efits.

### SUMMARY OF THE INVENTION

It would be highly desirable to formulate a stabilized arterial vasodilator/relaxant plus a platelet aggregation inhibitor combination oral composition of basic L-Arginine as a precursor biological source of the arterial epithelium relaxant nitric oxide and one or more of the aforementioned acidic “blood thinner” drugs that antagonize blood platelet clumping. Due to pharmaceutical and therapeutic instabilities of this combination composition, however, and the resultant potential gastrointestinal mucosal lesions caused by each of the aforementioned active drug substances, this has not been technically feasible.

It is an objective of this invention to provide a divalent magnesium ion stablilized gradual release oral composition of higly basic pH L-Arginine and the aforementioned “blood thinner” acid pH drug substance(s) as a method of preventing and treating cardiovascular arterial stenosis disease conditions including myocardium ischemia & heart attacks, cerebrovascular strokes and leg circulation disease conditions.

It is another objective of this invention to concurrently (1) dilate narrowed human arterial circulation by providing an oral biological active source of epithelial dependent relaxing factor and to (2) antagonize blood platelet aggregation in human vascular arteries, thereby helping reduce atherosclerotic plaques and increase blood flow through the coronary arteries to the heart myocardium, the carotid arteries to the brain and the femoral arteries to the leg musculature.

It is another objective of this invention to provide a gradual release oral composition of L-Arginine as a precursonr biological source of nitric oxide, the arterial epithelium dependent relaxing factor to dilate arteries narrowed due to cholesterol and aggregated platelet atherosclerotic plaques in a stabilized composition containing one or more platelet aggregation inhibitor drug substances aforementioned in this patent disclosure.

It is a further objective of this invention to provide a method of preventing gastrointestinal lesions resulting from oral administration of a combination of L-Arginine and one or more of the aforementioned platelet aggregation inhibitor drug substances on the basis of a gradual release composition utilizing a divalent magnesium ion additive to slow the release of the active drugs in the gastrointestinal tract and thereby prevent mucosal erosion. These and other objectives of this invention are provided and described in one or more of the embodiments presented as follows in the Description and Examples of the Preferred Embodiments of this invention.

### DESCRIPTION & EXAMPLES OF THE PREFERRED EMBODIMENTS

From the DESCRIPTION OF RELATED ART data presented herein, it is well established clinically that L-Arginine serves as a biological source of nitric oxide, the arterial epithelial derived relaxing factor EDRF to prevent and alleviate artery narrowing and thereby improve and maintain blood flow through the body's arteries. It is also well established clinically that several “blood thinner” drug substances presented herein are effective in inibiting or antagonizing platelet aggregation in cholesterolic atherosclerotic plaques in the arterial circulation that block blood flow resulting in cardiovascular diseases in the heart myocardium, in the brain causing strokes and in the leg musculature. Unfortunately, however, it has not been technically feasible to combine L-Arginine with one or more of the aforementioned platelet aggregation inhibitor drugs due to pharmaceutical and therapeutic incompatibility. The high alkaline pH of L-Arginine and the acid pH of these “blood thinner” drugs combined in a common oral composition can results in physico-chemical instability of one or more of the active substances and concurrent administration can cause gastrointestinal lesions when administered in therapeutic oral dosages.

The divalent magnesium ion stabilized compositions of this invention serve as a method to treat cardiovascular diseases by providing a combination of (1) L-Arginine as a biologically active precursor of nitric oxide released in the blood stream to relax and dilate the arteries to improve blood flow to the target organs and (2) one or more “blood thinner” drugs that prevent platelet aggregation and thereby help to reduce the cholesterolic-aggregated platelet atherosclerotic plaques that block arterial patency, thereby concurrently improving blood flow to the target organs by a second mechanism of action. “Blood thinner” agents therapeutically useful for this purpose include acetylsalicylic acid, clopidogrel hydrochloride, ticlopidine hydrochloride, anagrelide hydrochloride, pydridamole hydrochloride, and others in this pharmacological class of active drugs. Unforutunately, however, L-Arginine has a higly alkaline pH and the “blood thinners” are acid salts and therefore physico-chemically incompatible. We have unexpectedly found that by the linkage addition of a divalent magnesium salt, particularly mgnesium carbonate, although other divalent magnesium salts such as magnesium citrate, magnesium trisilicate, magnesium oxide and others in this chemical class may be used, it becomes possible to formulate stabilized oral compositions of the aforementioned active agents. In addition, the resultant compositions have the additional health benefit of effecting gradual release properties to the oral dosage form, thereby reducing the gastrointestinal erosion potential of the active agents released in-vivo in the body.

The oral drug compositions of this invention are best adminstered as compressed tablets manufactured on convention tabletting presses common in the pharmacetical industry. Examples to demonstrate the magnesium ion stabilizing mechanism and gradual release properties of the divalent magnesium ion linked with L-Arginine and “blood thinner’ drugs follow:

(1) Divalent Magnesium ion links the alkaline pH L-Arginine aminocarboxyl group and the acid pH Acetylsalicylic Acid carboxyl group in-vitro to form the pH neutral Magnesium linked stabilized complex that is slowly hydrolyzed in-vivo to L-Arginine and Magnesium acetylsalicylate

L-Arginine aminocarboxy-Mg-carboxy acetylsalicylate salt

(2) Oral dosage composition of L-Arginine and Acetylsalicylic acid plus Magnesium Carbonate

| | | |
|---|---|---|
| L-Arginine | 500 | milligrams |
| Acetylsalicylic Acid | 81 | milligrams |
| Magnesium Carbonate | 100 | mg eq. to 24 mg magnesium ion |
| Tablet Excipients | qs | ad 700 milligrams |

Disintegration: USP (701) Uncoated Tablets Procedure —2-3 hours gradual release

Stablity: 30 days stored at 45° C.—no physical or chemical changes (3) Divalent Magnesium ion links the alkaline pH L-Aginine aminocarboxyl group and the acid pH Clopidgrel Bisulfate pyridinoacetate group in-vitro to the pH neutral Magnesium linked stabilized complex that is slowly hydolyzed in-vivo to L-Arginine and the Magnesium salt of Clopidogrel Sulfate

L-Arginine aminocarboxy-Mg-pyridinoacetate bisulfate salt of Clopidogrel base

(4) Oral dosage composition of L-Arginine and Clopidogrel Bisulfate plus Magnesium Oxide

| | | |
|---|---|---|
| L-Arginine | 250 | milligrams |
| Clopidogrel Bisulfate | 75 | milligrams |
| Magnesium Oxide | 50 | mg eq. to 30 mg magnesium ion |
| Tablet Excip;ients | qs | ad 400 milligrams |

Disintegration: USP (701) Uncoated Tablets Procedure —2-3 hours gradual release

Stability: 30 days stored at 45° C.—no physical or chemical changes

(5) Divalent Magnesium ion links the alkaline pH L-Arginine aminocarboxyl group and the acid pH Anagrelide Hydrochloride quinazolin group in-vitro to the pH neutral Magnesium linked stablized complex that is slowly hydrolyzed in-vivo to L-Arginine and the Magnesium salt of Anagrelide Hydrochloride

L-Arginine aminocarboxy-Mg-quinazolin Hydrochloride salt of Analgrelide base

(6) Oral dosage composition of L-Arginine and Anagrelide Hydrochloride plus Magnesium Hydroxide

| | | |
|---|---|---|
| L-Arginine | 500 | milligrams |
| Anagrelide Hydrochloride | 5 | milligrams |
| Magnesium Hyroxide | 50 | mg equivalent to 21 milligrams |
| | | magnesium ion |
| Tablet Excipients | qs | ad 600 milligrams |

Disitegration USP (701) Uncoated Tablets Procedure 2-3 hours gradual release

Stability: 30 days stored at 45° C.—no physical or chemical changes

(7) Divalent Magnesium ion links the alkaline pH L-Arginine aminocarboxyl group and the acid pH Ticlopidine Hydrochloride chlorophenyl group in-vitro to the pH neutral Magnesium linked stabilized complex that is slowly hydrolyzed in-vivo to L-Arginine and the Magnesium salt of Ticlopidine Hydrochloride

(8) Oral dosage composition of L-Arginine and Ticlopidine Hydrochloride plus Magnesium Carbonate

| | | |
|---|---|---|
| L-Arginine | 250 | millligrams |
| Ticlopidine Hydrochloride | 125 | milligrams |
| Magnesium Carbonate | 75 | mg eq. to 18 milligrams |
| | | magnesium ion |
| Tablet excipients | qs | ad to 500 milligrams |

Disintegration USP (701) Uncoated Tablets Procedure 2-3 hours gradual release

Stability: 30 days stored at 45° C.—no physical or chemical changes

These composition examples are cited to demonstrate, but not to limit various concentrations of L-Arginine and “blood thinner” active ingredients in tablet excipient stabilized formulations to provide gradual release of the active ingredients in-vivo to the blood stream wherein L-Arginine forms the epithelial derived relaxation factor to dilate the arteries and the “blood thinner” ingredients work pharmacologically to inhibit platelet aggregation in atherosclerotic placques to improve blood flow through the arteries.

### DESCRIPTION OF RELATED ART

Cardiovascular, cerebrovascular, and leg circulation occlusion diseases occur when arteries become narrowed or occluded (known medically as stenosis) to the point where there is a reduced blood supply to the heart, brain and leg musculature respectively. Platelets are microscopic oval discs that circulate in the blood stream and normally serve an important function in blood clotting. However, individuals with high cholesterol tend to form arterial atherosclerotic plaques in which platelets also play a role to block normal blood flow through the arteries. Normally, platelets carry a negative charge to prevent clumping and adhering to arterial walls, but in cases of atherosclerosis, the platelets adhere to the cholesterol formed plaques that block the normal blood flow resulting in ischemia or blood deficiencies in the related organs. In the heart, blockage of the coronary arteries can result in infarcts or damaged heart muscle resulting in a heart attack; in the brain, blockage of the carotid arteries can result in strokes due to reduced oxygen supply to the brain; and in the legs, blockage of the femoral arteries can result in damage to the upper and lower leg muscles.

Several chemical substances have been medically used to provide an oral source of nitric oxide to the arterial circulation to relax the smooth musculature of arteries and maintain patency of the body's arteries. Among these are isosorbide mono and dinitrate, erythritol tetranitrtate, and L-Arginine. Chemically, L-Arginine is 2-amino-5-guanidino valeric acid, a highly basic amino acid, and is a preferred embodiment of this invention in that it serves as a dietary supplement source of nitric oxide in the body wherein L-Arginine is acted upon by the enzyme nitric oxide synthase to form nitric oxide. Nitric oxide has been shown clinically, as reported in the above cited referenced publications and patents, to effect arterial endothelium relaxation and dilation. This nitric oxide metabolite is known functionally as “EDRF”, the Endothelial Derived Relaxation Factor, and is well described in the cited literature and patent references. Medically, this amino acid L-Arginine plays an important beneficial cardiovascular health role to maintain the blood flow in the arterial circulation. Another separate group of drug substances known as “blood thinners” have been demonstrated clinically to prevent adhesion, clumping or aggregation of blood platelets, which in contact with elevated cholesterol levels in the blood, will form atherosclerotic plaques that block the normal flow of blood. The pathologic results of the resultant blockage or stenosis of the arteries are heart attacks in the case of coronary artery blockage; cerebrovascular strokes in the case of carotid artery blockage; and severe leg muscle damage in the case of femoral artery circulation blockage. Examples of these “blood thinner” drugs that inhibit the adhesion, clumping and aggregation of blood platelets are, but not limited to, acetylsalicylic acid, clopidogrel bisulfate, ticlopidine hydrochloride, anagrelide hydrochloride, pyridimole hydrochloride and the like. One type of “blood thinner” drugs typified by acetylsalicylic acid, serve as prostaglandin synthetase inhibitors, thereby preventing blood platelet aggregation and clotting by inhibiting the prostaglandin synthetase Thromboxane A2 (TxA2), the prostaglandin enzyme required for blood platelet aggregation and clotting. Another type of “blood thinner” drugs typified by clopidogrel bisulfate, ticlopidine hydrochloride and anagrelide hydrochloride serve as inhibitors of adenosine diphosphate (ADP) induced platelet aggregation by binding to the ADP receptor sites thereby interfering with ADP-collagen mediated platelet aggregation. Both these pharmacological class of “blood thinner” drugs are FDA approved as effective medically, and are marketed individually as commercial pharmaceutical products for the indicated cardiovascular health benefits.

### SUMMARY OF THE INVENTION

It would be highly desirable to formulate a stabilized arterial vasodilator/relaxant plus a platelet aggregation inhibitor combination oral composition of basic L-Arginine as a precursor biological source of the arterial epithelium relaxant nitric oxide and one or more of the aforementioned acidic “blood thinner” drugs that antagonize blood platelet clumping. Due to pharmaceutical and therapeutic instabilities of this combination composition, however, and the resultant potential gastrointestinal mucosal lesions caused by each of the aforementioned active drug substances, this has not been technically feasible.

It is an objective of this invention to provide a divalent magnesium ion stabilized gradual release oral composition of highly basic pH L-Arginine and the aforementioned “blood thinner” acid pH drug substance(s) as a method of preventing and treating cardiovascular arterial stenosis disease conditions including myocardium ischemia & heart attacks, cerebrovascular strokes and leg circulation disease conditions.

It is another objective of this invention to concurrently (1) dilate narrowed human arterial circulation by providing an oral biological active source of epithelial dependent relaxing factor and to (2) antagonize blood platelet aggregation in human vascular arteries, thereby helping reduce atherosclerotic plaques and increase blood flow through the coronary arteries to the heart myocardium, the carotid arteries to the brain and the femoral arteries to the leg musculature.

It is another objective of this invention to provide a gradual release oral composition of L-Arginine as a precursor biological source of nitric oxide, the arterial epithelium dependent relaxing factor to dilate arteries narrowed due to cholesterol and aggregated platelet atherosclerotic plaques in a stabilized composition containing one or more platelet aggregation inhibitor drug substances aforementioned in this patent disclosure.

It is a further objective of this invention to provide a method of preventing gastrointestinal lesions resulting from oral administration of a combination of L-Arginine and one or more of the aforementioned platelet aggregation inhibitor drug substances on the basis of a gradual release composition utilizing a divalent magnesium ion additive to slow the release of the active drugs in the gastrointestinal tract and thereby prevent mucosal erosion. These and other objectives of this invention are provided and described in one or more of the embodiments presented as follows in the Description and Examples of the Preferred Embodiments of this invention.

### DESCRIPTION & EXAMPLES OF THE PREFERRED EMBODIMENTS

From the DESCRIPTION OF RELATED ART data presented herein, it is well established clinically that L-Arginine serves as a biological source of nitric oxide, the arterial epithelial derived relaxing factor EDRF to prevent and alleviate artery narrowing and thereby improve and maintain blood flow through the body's arteries. It is also well established clinically that several “blood thinner” drug substances presented herein are effective in inhibiting or antagonizing platelet aggregation in cholesterolic atherosclerotic plaques in the arterial circulation that block blood flow resulting in cardiovascular diseases in the heart myocardium, in the brain causing strokes and in the leg musculature. Unfortunately, however, it has not been technically feasible to combine L-Arginine with one or more of the aforementioned platelet aggregation inhibitor drugs due to pharmaceutical and therapeutic incompatibility. The high alkaline pH of L-Arginine and the acid pH of these “blood thinner” drugs combined in a common oral composition can results in physico-chemical instability of one or more of the active substances and concurrent administration can cause gastrointestinal lesions when administered in therapeutic oral dosages.

The divalent magnesium ion stabilized compositions of this invention serve as a method to treat cardiovascular diseases by providing a combination of (1) L-Arginine as a biologically active precursor of nitric oxide released in the blood stream to relax and dilate the arteries to improve blood flow to the target organs and (2) one or more “blood thinner” drugs that prevent platelet aggregation and thereby help to reduce the cholesterolic-aggregated platelet atherosclerotic plaques that block arterial patency, thereby concurrently improving blood flow to the target organs by a second mechanism of action. “Blood thinner” agents therapeutically useful for this purpose include acetylsalicylic acid, clopidogrel hydrochloride, ticlopidine hydrochloride, anagrelide hydrochloride, pydridamole hydrochloride, and others in this pharmacological class of active drugs. Unfortunately, however, L-Arginine has a highly alkaline pH and the “blood thinners” are acid salts and therefore physico-chemically incompatible. We have unexpectedly found that by the linkage addition of a divalent magnesium salt, particularly magnesium carbonate, although other divalent magnesium salts such as magnesium citrate, magnesium trisilicate, magnesium oxide and others in this chemical class may be used, it becomes possible to formulate stabilized oral compositions of the aforementioned active agents. In addition, the resultant compositions have the additional health benefit of effecting gradual release properties to the oral dosage form, thereby reducing the gastrointestinal erosion potential of the active agents released in-vivo in the body.

The oral drug compositions of this invention are best administered as compressed tablets manufactured on convention tabletting presses common in the pharmaceutical industry. Examples to demonstrate the magnesium ion stabilizing mechanism and gradual release properties of the divalent magnesium ion linked with L-Arginine and “blood thinner’ drugs follow:

(1) Divalent Magnesium ion links the alkaline pH L-Arginine aminocarboxyl group and the acid pH Acetylsalicylic Acid carboxyl group in-vitro to form the pH neutral Magnesium linked stabilized complex that is slowly hydrolyzed in-vivo to L-Arginine and Magnesium acetylsalicylate—L-Arginine aminocarboxy-Mg-carboxy acetylsalicylate salt

(2) Oral dosage composition of L-Arginine and Acetylsalicylic acid plus Magnesium Carbonate

| | | |
|---|---|---|
| L-Arginine | 500 | milligrams |
| Acetylsalicylic Acid | 81 | milligrams |
| Magnesium Carbonate | 100 | mg eq. to 24 mg magnesium ion |
| Tablet Excipients | qs | ad 700 milligrams |

Disintegration: USP (701) Uncoated Tablets Procedure —2-3 hours gradual release

Stability: 30 days stored at 45° C.—no physical or chemical changes

(3) Divalent Magnesium ion links the alkaline pH L-Arginine aminocarboxyl group and the acid pH Clopidgrel Bisulfate pyridinoacetate group in-vitro to the pH neutral Magnesium linked stabilized complex that is slowly hydrolyzed in-vivo to L-Arginine and the Magnesium salt of Clopidogrel Sulfate—L-Arginine aminocarboxy-Mg-pyridinoacetate bisulfate salt of Clopidogrel base.

(4) Oral dosage composition of L-Arginine and Clopidogrel Bisulfate plus Magnesium Oxide

| | | |
|---|---|---|
| L-Arginine | 250 | milligrams |
| Clopidogrel Bisulfate | 75 | milligrams |
| Magnesium Oxide | 50 | mg eq. to 30 mg magnesium ion |
| Tablet Excipients | qs | ad 400 milligrams |

Disintegration: USP (701) Uncoated Tablets Procedure —2-3 hours gradual release

Stability: 30 days stored at 45° C.—no physical or chemical changes

(5) Divalent Magnesium ions links the alkaline pH L-Arginine amonocarboxl group and the acid pH Anagrelide Hydrochloride quinazolin group in-vitro to the pH neutral Magnesium linked stabilized complex that is slowly hydrolyzed in-vivo to L-Arginine and the Magnesium salt of Anagrelide Hydrochloride—L-Arginine aminocarboxy-Mg-quinazolin Hydrochloride salt of Analgrelide base

(6) Oral dosage composition of L-Arginine and Anagrelide Hydrochloride plus Magnesium Hydroxide

| | | |
|---|---|---|
| L-Arginine | 500 | milligrams |
| Anagrelide Hydrochloride | 5 | milligrams |
| Magnesium Hydroxide | 50 | mg equivalent to 21 milligrams |
| | | magnesium ion |
| Tablet Excipients | qs | ad 600 milligrams |

Disintegration USP (701) Uncoated Tablets Procedure 2-3 hours gradual release

Stability: 30 days stored at 45° C.—no physical or chemical changes

(7) Divalent Magnesium ion links the alkaline pH L-Arginine aminocarboxyl group and the acid pH Ticlopidine Hydrochloride chlorophenyl group in-vitro to the pH neutral Magnesium linked stabilized complex that is slowly hydrolyzed in-vivo to L-Arginine and the Magnesium salt of Ticlopidine Hydrochloride

(8) Oral dosage composition of L-Arginine and Ticlopidine Hydrochloride plus Magnesium Carbonate

| | | |
|---|---|---|
| L-Arginine | 250 | milligrams |
| Ticlopidine Hydrochloride | 125 | milligrams |
| Magnesium Carbonate | 75 | mg eq. to 18 milligrams |
| | | magnesium ion |
| Tablet excipients | qs | ad to 500 milligrams |

Disintegration USP (701) Uncoated Tablets Procedure 2-3 hours gradual release

Stability: 30 days stored at 45° C.—no physical or chemical changes

These composition examples are cited to demonstrate, but not to limit various concentrations of L-Arginine and “blood thinner” active ingredients in tablet excipient stabilized formulations to provide gradual release of the active ingredients in-vivo to the blood stream wherein L-Arginine forms the epithelial derived relaxation factor to dilate the arteries and the “blood thinner” ingredients work pharmacologically to inhibit platelet aggregation in atherosclerotic plaques to improve blood flow through the arteries.

What is claimed is:

1. Stabilized oral drug compositions consisting of the active ingredients L-Arginine, a biological source of nitric oxide that dilates arteries, and is chemically 2-mono-5-guanidino valeric acid, crosslinked with a divalent magnesium salt and a blood platelet aggregation inhibitor compound such as acetylsalicylic acid, clopidogrel bisulfate, ticlopidine hydrochloride, anagrelide hydrochloride, pyridamole hydrochloride, and the like that antagonize blood platelet aggregation resulting in reduced atherosclerotic plaques and improved arterial patency with resultant increased blood flow to the myocardium in the case of coronary arteries, to the brain in the case of carotid arteries and to the femoral arteries in the case of leg circulation stenosis.

2. Drug compositions containing the ingredients in claim 1 wherein the said compositions are physically and chemically stabilized by the divalent magnesium salt to extend shelf-life of the compositions to maintain their cardioprotective health benefits of improved blood flow through the coronary arteries to the heart muscle, the carotid arteries to the brain and the femoral arteries to the leg musculature.

3. Oral drug compositions containing the ingredients in claim 1 wherein the active ingredients are gradually released in the body to reduce the potential for producing gastrointestinal lesions while providing the cardioprotective health benefits of improved blood flow through the coronary arteries to the heart muscle, through the carotid arteries to the brain and through the femoral arteries to the leg musculature.

4. Oral drug compositions consisting of the active ingredients L-Arginine, chemically 2-mono-5-guanidino valeric acid, and a blood platelet aggregation inhibitor compound such as acetylsalicylic acid, clopidogrel bisulfate, ticlopidine hydrochloride, anagrelide hydrochloride, pyridamole hydrochloride, and the like stabilized with a divalent magnesium salt such as magnesium carbonate, magnesium hydroxide, magnesium oxide and the like to improve physical and chemical stability of the active ingredients and to allow gradually release of the active ingredients in the gastrointestinal tract to protect the gastrointestinal lining from the erosion potential of the composition active ingredients.

5. Stabilized oral drug compositions consisting of the active ingredients L-Arginine, a biological source of nitric oxide and chemically 2-mono-5-guanidino valeric acid crosslinked with a divalent magnesium salt and the blood platelet aggregation inhibitor acetylsalicylic acid wherein the magnesium ion binds with the decomposition product acetic acid to stabilize the composition, and wherein the magnesium ion neutralizes the highly alkaline pH of L-Arginine and the highly acid pH of acetylsalicylic acid to protect the gastrointestinal mucosa from erosion.

6. Stabilized oral drug compositions consisting of the active ingredients L-Arginine, a biological source of nitric oxide and chemically 2-mono-5-guanidino valeric acid crosslinked with a divalent magnesium salt and the blood platelet aggregation inhibitor clopidogrel bitartrate, chemically methyl-S-alpha-2-chlorophenol 6-7 dihydrothienol pyridine acetate sulfate, wherein the magnesium ion neutralizes the highly alkaline pH of L-Arginine and the acidic pH of clopidogrel bitartrate to protect the gastrointestinal mucosa from erosion.

7. Stabilized oral drug compositions of claims 5 and 6 consisting of the active ingredients L-Arginine crosslinked with a divalent magnesium salt and a blood platelet aggregation inhibitor compound such as ticlopidine hydrochloride, analgrelide hydrochloride, pyridamole hydrochloride, and the like wherein the magnesium ion neutralizes the highly alkaline pH of L-Arginine and the highly acidic pH of the blood platelet aggregation inhibitor compounds to protect the gastrointestinal mucosa from erosion.

8. A method of reducing atherosclerotic plaques in the human arteries with gradual release oral drug compositions to protect the gastrointestinal mucosa consisting of L-Arginine, a biological source of nitric oxide released in the coronary, carotid and femoral artery epithelium as the endothelium dependent relaxing factor EDRE to dilate the arteries to promote blood flow to the myocardium, to the brain and to the legs respectively, and a blood platelet aggregation inhibitor compound described in claim 1 wherein the L-Arginine and blood platelet aggregation inhibitor composition is stabilized with a divalent magnesium salt described in claim 4.

9. A method of treating cardiovascular disease with stabilized oral compositions consisting of L-Arginine, a biological source of nitric oxide released in the coronary artery epithelium as the endotheliium dependent relaxing factor EDRF to dilate the arteries to promote blood flow to the myocardium, to the brain, and to the leg musculature, and a blood platelet aggregation inhibitor compound described in claim 1 that reduces arterial atherosclerotic plaques and wherein the L-Arginine and blood platelet aggregation inhibitor composition is stabilized with a divalent magnesium salt described in claim 4.

10. A method of treating cardiovascular disease with gradual release oral compositions consisting of L-Arginine and a blood platelet aggregation inhibitor compound described in claim 1 crosslinked and stabilized with a divalent magnesium salt described in claim 4 wherein the L-Arginine and the blood platelet aggregation inhibitor are slowly released in the gastrointestinal tract to prevent mucosal erosion.

11. A method of treating coronary artery disease with gradual release oral compositions consisting of L-Arginine and a blood platelet aggregation inhibitor compound described in claim 1 crosslinked and stabilized with a divalent magnesium salt described in claim 4 wherein the L-Arginine and the blood platelet aggregation inhibitor are slowly released in the gastrointestinal tract to prevent mucosal erosion.

12. A method of treating cerebrovascular accidents such as stroke with gradual release oral compositions of L-Arginine and a blood platelet aggregation inhibitor compound described in claim 1 crosslinked and stabilized with a divalent magnesium salt described in claim 4 wherein the L-Arginine and the blood platelet aggregation inhibitor are slowly released in the gastrointestinal tract to prevent mucosal erosion.

13. A method of treating leg arterial circulation occlusion with gradual release oral compositions of L-Arginine and a blood platelet aggregation inhibitor compound described in claim 1 cross-lined and stabilized with a divalent magnesium salt described in claim 4 wherein the L-Arginine and the blood platelet aggregation inhibitor are slowly released in the gastrointestinal tract to prevent mucosal erosion.
